(19) United States
(12) Patent Application Publication
(10) Pub. No.: US 2022/0163541 A1
(43) Pub. Date: May 26, 2022

(54) METHODS OF DIAGNOSING AND TREATING MULTIPLE SCLEROSIS WITH VASCULAR ENDOTHELIAL BIOMARKERS

(71) Applicant: Board of Supervisors of Louisiana State University and Agricultural and Mechanical College, Baton Rouge, LA (US)

(72) Inventors: Johnathan Steven ALEXANDER, Shreveport, LA (US); Christopher KEVIL, Shreveport, LA (US); Elizabeth DISBROW, Shreveport, LA (US); Karen Y. STOKES, Shreveport, LA (US); J. Winny YUN, Shreveport, LA (US); Pooja VEERAREDDY, Shreveport, LA (US); Urska Cvek, Shreveport, LA (US)

(73) Assignee: Board of Supervisors of Louisiana State University and Agricultural and Mechanical College, Baton Rouge, LA (US)

(21) Appl. No.: 17/535,982

(22) Filed: Nov. 26, 2021

Related U.S. Application Data
(60) Provisional application No. 63/118,028, filed on Nov. 25, 2020.

(51) Int. Cl. G01N 33/68; G01N 33/573; C12N 9/10; C12N 9/88

(52) U.S. Cl. CPC G01N 33/6896; G01N 33/573; C12N 9/1085; C12N 9/88; C12Y 208/01002; C12Y 205/01; G01N 2800/285; C12Y 404/01001; C12N 9/13

(57) ABSTRACT

The presently disclosed invention relates to a method of diagnosing Multiple Sclerosis (MS) in a patient comprising obtaining a sample from the patient, determining a level of one or more H2S generating enzymes in the sample from the patient, and diagnosing the patient with MS when the level of the one or more H2S generating enzymes is one of at least 15% higher or lower than a control level for the one or more H2S generating enzymes, and at least 10% higher or lower than a control level for the one or more H2S generating enzymes.

### CROSS REFERENCE TO RELATED APPLICATIONS/PRIORITY

The present invention claims priority to U.S. Provisional Patent Application No. 63/118,028 filed Nov. 25, 2020, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. P20 GM121307 awarded by the National Institutes of Health. The government has certain rights in the invention.

### BACKGROUND

Multiple Sclerosis (MS) is the leading cause of permanent neurologic disability in young adults. The inventors observe that MS is not just a group of immune-mediated central nervous system (CNS) demyelinating diseases, but one of a group of vascular neuroinflammatory conditions. The inventors offer that the complex pathogenesis of MS can only be appreciated when and if its vascular contributions are recognized as significant features of all forms of MS etiology. The inventors propose that disturbances in the expression and organization of ‘neurolymphatic’/‘glymphatic’ features of blood brain barrier (BBB) vessels represent extremely important and mechanism-based but under-recognized characteristics of the MS inflamed cerebrum that contributed to progression and intensification of the MS with the brain endothelial cells as the ‘failing gatekeeper’. The assumption of the industry has been that the presence of neurolymphatic markers in blood reflected a brain-endothelial origin because of the demonstration of these proteins in human and brain endothelial cells and their supernatants, changes in the abundance and shedding of these proteins following challenge with inflammatory cytokines, the intimate endothelial surface interaction with the circulating serum/plasma and the blood and the initial compartmentalization of these lymphatic biomarkers into ‘microparticles’ (MP).

Because of the long-held perception that the CNS lacks conventional lymphatics, studies on lymphatic involvement in MS pathogenesis have largely neglected roles brain lymphatics might play in eliminating inflammatory mediators in the CNS. The inventors are aware that CNS interstitial fluid and solutes can drain along 150-200 nm wide ‘lacunae’ in the basement membranes of arteries and capillary walls, representing a conduit for elimination of brain interstitial/neurolymphatic contents, and that a brain-wide system of channels facilitate the clearance of interstitial components from the brain which are termed the ‘glymphatic’ system. So far, the molecular identities and cellular origins of these networks remain uncharacterized. The inventors argue that the networks appear to represent important diagnostic, staging and therapeutic targets in neurodegenerative diseases.

Presently, putative MS-patients are only diagnosed with MS after at least two separate areas of the CNS are found to be damaged by MRI imaging. Only then will physicians employ various neurologic exams and lab tests to rule out other diseases to diagnose MS. These lab tests are often complicated and invasive, involving lumbar punctures to obtain and evaluate CSF samples, and forestalls accurate diagnosis until the disease has progressed, increasing severity of the disease. For the foregoing reasons, there is a pressing, but seemingly irresolvable need for accurate, inexpensive, non-obtrusive diagnostic tests for MS.

### SUMMARY

Wherefore, it is an object of the present invention to overcome the above mentioned shortcomings and drawbacks associated with the current technology. The present invention is directed to methods and apparatuses for testing and treating MS.

The presently disclosed invention relates to a method of diagnosing Multiple Sclerosis (MS) in a patient comprising obtaining a sample from the patient, determining a level of one or more H2S generating enzymes in the sample from the patient, and diagnosing the patient with MS when the level of the one or more H2S generating enzymes is one of at least 15% higher or lower than a control level for the one or more H2S generating enzymes, and at least 10% higher or lower than a control level for the one or more H2S generating enzymes. According to a further embodiment the patient is diagnosed with MS if one or more H2S generating enzymes is one of Cystathionine g-lyase (CSE), cystathionine b-synthase (CBS), and 3-mercaptopyruvate sulfurtransferase (MST). According to a further embodiment one of the one or more H2S generating enzymes is CSE, the patient is diagnosed with MS if the level of CSE in the sample is 10-20% higher than a control level, the one or more H2S generating enzymes is CBS, the patient is diagnosed with MS if the level of CBS in the sample is 15-25% lower than a control level, and the one or more H2S generating enzymes is MST, the patient is diagnosed with MS if the level of MST in the sample is a 15-25% lower than a control level. According to a further embodiment the patient is diagnosed with MS when both (a) the one or more H2S generating enzymes includes two of CSE, CBS, and MST, and (b) two of the following occur the level of CSE in the sample is 10-20% higher than a control level, the level of CBS in the sample is 15-25% lower than a control level, and the level of MST in the sample is a 15-25% lower than a control level. According to a further embodiment the patient is diagnosed with MS when each of the one or more H2S generating enzymes includes two of CSE, CBS, and MST, the level of CSE in the sample is 10-20% higher than a control level, the level of CBS in the sample is 15-25% lower than a control level, and the level of MST in the sample is a 15-25% lower than a control level. According to a further embodiment the sample is one of plasma, blood, urine, sputum or saliva. According to a further embodiment, the method further comprises centrifugally isolating extracellular vesicles and microparticles from the sample to determine the level of the one or more H2S generating enzymes. According to a further embodiment, the method further comprises centrifuging the sample at 20,000×g for 1 h and analyzing a formed pelleted material for one or more H2S generating enzymes.

The presently disclosed invention further relates to a method of diagnosing and treating Multiple Sclerosis (MS) in a patient comprising obtaining a sample from the patient; determining a level of one or more H2S generating enzymes in the sample from the patient; diagnosing the patient with MS when the level of the one or more H2S generating enzymes in the sample is one of at least 15% higher or lower than a control level for the one or more H2S generating enzymes, and at least 10% higher or lower than a control level for the one or more H2S generating enzymes, and administering an effective amount of one of an H2S generating enzyme inhibitor to the diagnosed patient if the one or more H2S generating enzymes level in the sample is elevated, and additional H2S generating enzyme substrate and or H2S generating enzyme genetic augmentation to the diagnosed patient if the one or more H2S generating enzymes level in the sample is decreased. According to a further embodiment the sample is one of plasma, blood, urine, sputum or saliva.

The presently disclosed invention further relates to a method of treating a patient having Multiple Sclerosis (MS) patient in need of treatment comprising administering a pharmaceutically effective dose of a therapeutic, wherein the therapeutic includes one of an H2S generating enzyme inhibitor, additional H2S generating enzyme substrate, and H2S generating enzyme genetic augmentation. According to a further embodiment the one or more H2S generating enzymes includes CSE, and the therapeutic includes a CSE inhibitor. According to a further embodiment the one or more H2S generating enzymes includes CBS, and the therapeutic includes one of a CBS substrate and CBS genetic augmentation. According to a further embodiment the one or more H2S generating enzymes includes MST, and the therapeutic includes one of a MST substrate and MST genetic augmentation. According to a further embodiment (a) the one or more H2S generating enzymes includes each of CSE, CBS, and MST, and (b) the therapeutic includes each (i) a CSE inhibitor, (ii) one of a CBS substrate and CBS genetic augmentation, and (iii) one of a MST substrate and MST genetic augmentation. According to a further embodiment, the method further includes administration of a further therapeutic. According to a further embodiment the further therapeutic includes one of a biologic and immunomodulatory therapy. According to a further embodiment, the further therapeutic includes one of Copaxone, Gilenya, Tecfidera, Tysabri, Aubagio, Rebif, Ampyra, Ocrevus, Avonex, teriflunomide, Plegridy, Mavenclad, Mayzent, ozanimod, Vumerity, Zeposia, prednisone, Betaseron, Avonex Pen, glatiramer, dalfampridine, fingolimod, interferon beta-1a, dexamethasone, dimethyl fumarate, Lemtrada, natalizumab, vedolizumab, steroids, ocrelizumab, Acthar, Extavia, Imuran, valacyclovir Off Label, azathioprine, Dexamethasone Intensol, interferon beta-1b, methylprednisolone, prednisolone, Rebif Rebidose, Solu-Medrol, alemtuzumab, cladribine, Glatopa, mitoxantrone, mycophenolate mofetil Off Label, Azasan, corticotropin, cyclophosphamide Off Label, daclizumab, De-Sone LA, Dxevo, H.P. Acthar Gel, HiDex, Medrol, Millipred, Millipred DP, Orapred ODT, PediaPred, peginterferon beta-1a, siponimod, Bafiertam, diroximel fumarate, Kesimpta, monomethyl fumarate, and ofatumumab.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a box plot which shows CSE and Ponceau Normalized Data;

FIG. 2 is a box plot which shows CBS and Ponceau Normalized Data ** indicates p≤0.01; and

FIG. 3 is a box plot which shows MST and Ponceau Normalized Data. **** indicates p≤0.0001.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means that the property is within 80% of its desired value. In other embodiments, “substantially” means that the property is within 90% of its desired value. In other embodiments, “substantially” means that the property is within 95% of its desired value. In other embodiments, “substantially” means that the property is within 99% of its desired value. For example, the term “substantially complete” means that a process is at least 80% complete, for example. In other embodiments, the term “substantially complete” means that a process is at least 90% complete, for example. In other embodiments, the term “substantially complete” means that a process is at least 95% complete, for example. In other embodiments, the term “substantially complete” means that a process is at least 99% complete, for example.

The term “substantially” includes a value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

The term “about” includes when value is within about 10% of the indicated value. In certain embodiments, the value is within about 5% of the indicated value. In certain embodiments, the value is within about 2.5% of the indicated value. In certain embodiments, the value is within about 1% of the indicated value. In certain embodiments, the value is within about 0.5% of the indicated value.

In addition, the invention does not require that all the advantageous features and all the advantages of any of the embodiments need to be incorporated into every embodiment of the invention.

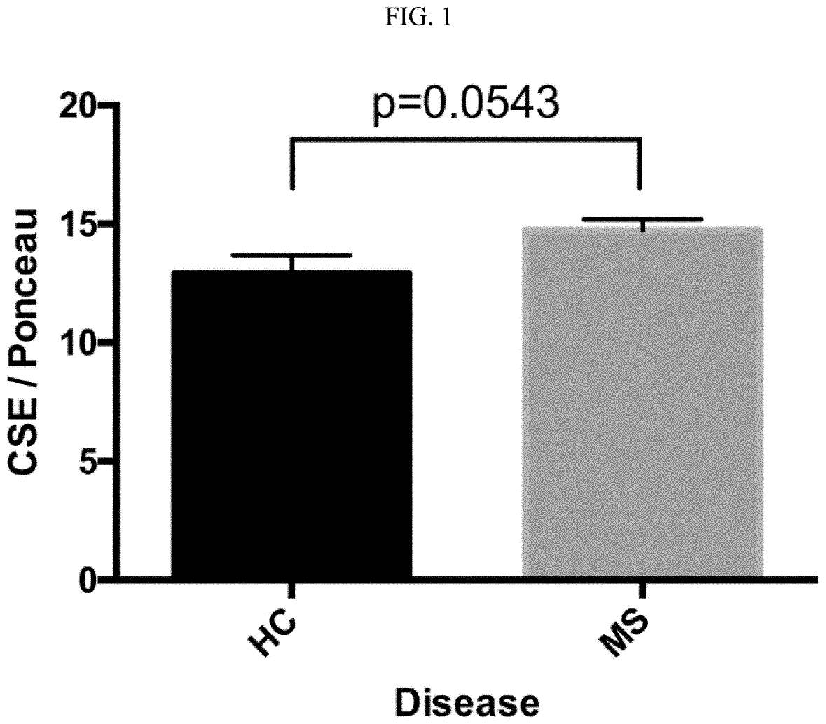

Turning now to FIGS. 1-3, a brief description concerning the various components of the present invention will now be briefly discussed.

Cystathionine g-lyase (CSE), cystathionine b-synthase (CBS), and 3-mercaptopyruvate sulfurtransferase (MST) are considered the three principal mammalian H₂S (hydrogen sulfide) generating enzymes. Findings in this disclosure reveal novel relationships with H₂S producing enzymes, CSE, CBS, and MST and increased levels of sulfide within plasma/serum from patients with Multiple sclerosis. Sulfide metabolites of these enzymes could potentially be a cause of neurological and vascular dysfunction. This work developed based off of evidence that H₂S gasotransmitter metabolites-sulfides are dysregulated in Alzheimer's disease and related dementias. Levels of these products was significantly and inversely correlated with cognitive and cerebral vascular function. Findings of a correlation in increased sulfide levels and endothelial dysfunction reveal a unique and novel approach to identify and stage individuals with Multiple sclerosis and also serve as a target for future therapeutic interventions. A result of the discovery disclosed herein is that H2S generating enzyme levels are dysregulated in MS. A treatment of the overall condition of an MS patient would then be to correct the hypo or hyper-active enzyme levels to treat what is either a symptom of MS or a cause of MS, but a condition associated with MS nonetheless. For H2S generating enzyme at increased levels, proper treatment may include administering a H2S generating enzyme inhibitor. For H2S generating enzyme at decreased level, proper treatment may include administering to the patient additional substrate and/or genetic augmentation. Genetic augmentation may include, for example, genetically supplement, adensoens deaminase, and crisp cas-9.

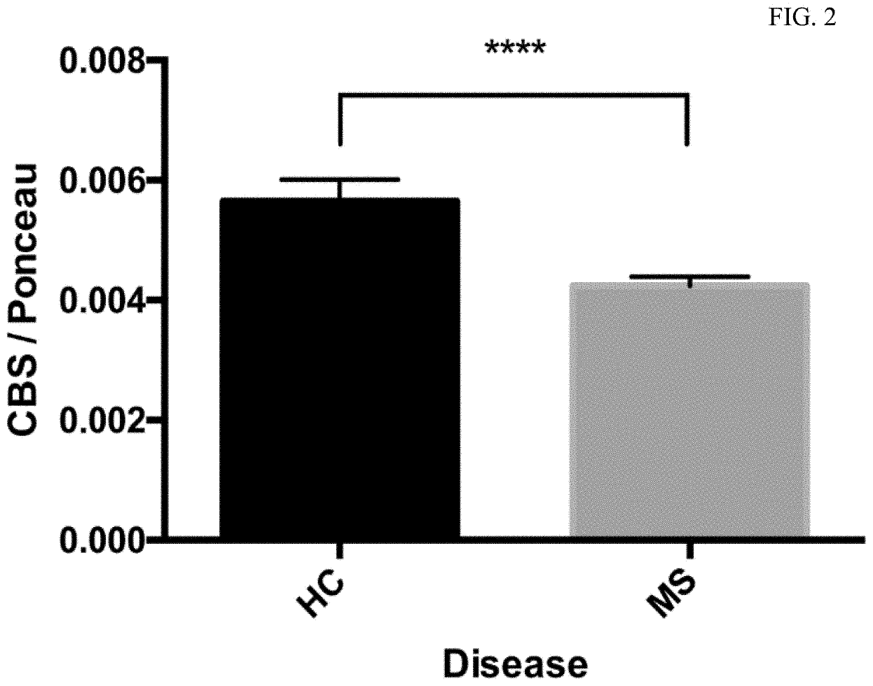

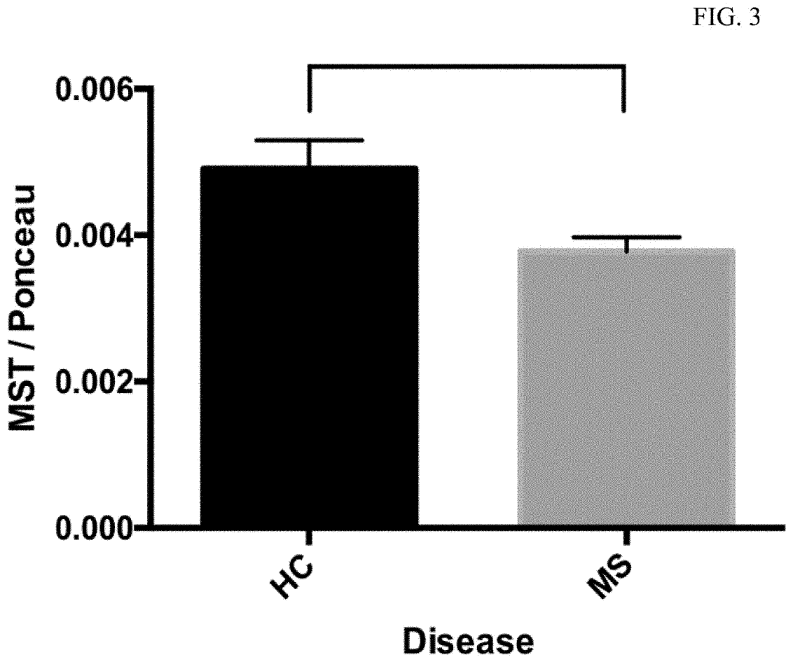

This study evaluated changes in oxidant and antioxidant enzymes (CSE, CBS, and MST) in human brain endothelial cells and tissue samples of patients with MS to determine biomarkers for the initiation and disease progression of Multiple sclerosis. The inventors hypothesized that these cytosolic and mitochondrial enzymes are responsible for producing sulfides in patients and that there would be differences in the control and MS samples based off evidence of increased H₂S levels being attributed to CSE in ADRD patients. Dot Blot analysis was performed with 260 MS and control serum Samples. The data below are representative of aggregate results from this analysis, combining secondary progressive MS (SPMS) and relapsing-remitting MS (RRMS) groups. Compared to control group serum levels, MS group serum levels showed a 10-20% increase in CSE (FIG. 1), a 15-25% decrease in CBS (FIG. 2), and a 15-25% decrease in MST (FIG. 3).

The presently claimed invention further includes the use of western blotting (WB), fluorescence-activated cell sorting (FACS) or enzyme-linked immunosorbent analysis (ELISA), for example, to analyze, diagnose, stage neurodegenerative and dementia diseases and to gauge the therapeutic efficacy of treatments for these diseases using a novel panel of circulating soluble proteins released by brain endothelial cells in microparticles' which are secreted ‘caveolar’ membrane-microdomains. The inventors have experimentally confirmed that the circulating depot of these biomarkers are initially liberated in small (<1 μm) microparticles which in this setting are brain endothelial membrane-derived shed caveolae (cholesterol-enriched membrane ‘blebs’. Microparticles are continuously released by endothelial cells and the rate of microparticle release is increased by exposure to conditions which are found in chronic inflammatory states including multiple sclerosis (MS). The inflammatory milieu in these conditions is characterized by an altered level of H2S generating enzymes. Because lymphatics and neurolymphatics function to clear disease modifying proteins and mediators like cytokines and beta-amyloid, the suppression of lymphatics, measured as a diminution of lymphatic structural and lineage-determining transcription factors would contribute to lymphostasis/neurolymph-stasis which fails to clear these injurious substances from the CNS and intensify disease activity.

The inventors disclose H2S generating enzymes both as a non-centrifugable freely-circulating pool and also a pool which is associated with microparticles which can be harvested by 10,000 g centrifugation. Both pools are found in and seen to be modulated by cytokine-stimulation in culture supernatants from human and mouse brain endothelial cells as well as in both control and multiple sclerosis serum samples which also show differences in these markers. Because microparticles are cleared from the vascular space by the reticuloendothelial system within hours of the time they are formed, they appear to represent a circulating recent ‘snapshot’ of the surface of the vasculature which describes the inflammatory complexion of the vasculature. In this manner, sampling of this pool is very simple and relative serum abundance of the H2S generating enzymes can be accomplished with as little as 0.25 μl of serum, indicating that a fingertip draw could provide sufficient sample for analysis. In this manner, it would be possible to repeatedly measure these marker enzyme in patients, potentially adopting scale technology currently used in diabetes testing approaches. This would allow simple, safe and repeated testing and tracking of a biomarker panel which may establish individual baseline profiles, measure responses to therapy and potentially anticipate disease intensifications. Additionally, these lymphatic biomarkers may also be tested using immunoanalysis (as described below) in plasma, cerebrospinal fluid, CSF, saliva, urine and tears and other biological fluids. In the event that patient shows an increased CSE level and/or an decreased CBS level and/or an decreased MST level, in addition to a suspected diagnosis of multiple sclerosis, the patient might be started earlier on biologic and immunomodulatory therapies e.g. natalizumab, vedolizumab and steroids. This is a critical advantage since it is widely known that earlier treatment to arrest disease activity can potently suppress the aggressive progression of the disease.

The release of microparticles is increased in response to inflammatory cytokine mediators, transferring these enzymes into a circulating pool, which is analyzed as a ‘liquid biopsy’ of the vascular endothelial surface to provide important information on the state of activation and inflammation within the brain. The inventors have studied these markers and found that over storage, markers which are originally restricted to microparticles are liberated into the soluble, non-centrifugable fraction which simplifies their analysis.

Current tests for a great many diseases, often rely on the robustness of a single biomarkers specificity and sensitivity. This means that any biomarker preferably varies closely with disease activity and preferably does not fluctuate independent of disease status, which could potentially invalidate the utility of the test. The use of multiple transcription factors, proteins which govern the expression of structural and signaling biomarkers provide information regarding how close to a particular cell lineage (or state of differentiation) a tissue may be under a set of conditions. In this approach, using two or three H2S generating enzymes may predict the ability of neurolymphatic networks to clear inflammatory stimuli. If lymphatic networks lose their appropriate differentiated structure/function changes in neurolymphatics could diagnose MS, predict ‘flares’ and gauge the efficacy of treatments to treat such disease.

Current versions of this approach anticipate that alteration of H2S generating enzymes level would be useful analytes revealing evidence of brain endothelial stress induced by inflammatory cytokines.

Release of apical microparticles (AMP) shedding by brain endothelial cells is time and cytokine concentration dependent. Endothelial cells continuously release MP and the rate of this release is increased by both TNF and IFN in a concentration dependent manner. This demonstrates that while NEBULA markers are associated with microparticles in plasma, samples of serum promote microparticle ‘fission’ confirmed by centrifugation recovery of caveolin-1 in plasma whereas this marker is also liberated in serum and is only found in the liquid phase.

Inventors' data show that progressive microparticle fission quantitatively and substantially or effectively completely transfers biomarkers into serum. Therefore, stored and frozen serum is a more superior, stable and representative analyte than plasma which should be employed in this analytical approach. Interestingly, as little as 0.25 μl of serum samples prepared in this manner provides sufficient material for biomarker testing, making this a highly robust approach.

The progression from microparticle (MP) shedding by brain endothelial cells, where MPs containing neurolymphatic biomarkers are shed following exposure to cytokine. These MPs arrive immediately in the plasma pool which can be centrifuged; it is important to note that use of plasma microparticles could lead to varying sample recovery depending on the time of centrifugation after recovery. By comparison, serum represents a much more stable pool for analysis.

The inventors disclose a single or multi marker develop diagnostic/prognostic test for MS.

Because several H2S generating enzyme levels or markers can be simultaneously analyzed, each individual component of the multi-analyte panel contributes to the creation of an algorithm known as a ‘principal component analysis’ (PCA), which provides greater predictive power over an individual specie. This approach has been enabled by the inventors and is adaptable for automated scoring and interpretation of these test results. This method is immediately useful to diagnose MS. The testing algorithm becomes even more predictive with two and then all three markers are considered. Multiple lower serum levels dramatically increases the reliability of the diagnosis.

The findings disclosed support and validate a concept that brain endothelial cells basally express apparently all known lymphatic endothelial cell biomarkers, many of which are recoverable and detectable within microparticles shed from inflammation-activated endothelium. This provides a liquid snapshot of the luminal surface of the brain vasculature during inflammatory phenomena and constitutes a novel means of evaluating ex vivo the level of inflammatory activation of the neurovasculature.

The disclosed findings represent a collective, multi-component ‘biomarker’ which has enormous commercial value to companies developing tests for MS and which could provide a mechanism-based test. This approach might be a secondary test to confirm or validate magnetic resonance imaging (MRI) results, or a primary test for these conditions subsequent to appropriate testing. Drug companies testing the efficacy of biologics and immune modulators for human therapy would be interested in the availability of a simple test to explore how and which symptoms of MS track with which markers. The approach also uses an unbiased computer interpretation to create the PCA test and has higher through-put than film reading in some respects.

In one embodiment, a testing kit based on this invention, would preferably include one or more of an immobiling phase e.g. a piece of nitrocellulose, to which a sample would be applied. This would be dried and blocked with 5% bovine serum albumin for 1 h and then incubated with an anti-H2S generating enzyme antibody, such as an anti-CBS antibody, at a dilution of, for example, 1:1000 in 0.1% bovine serum albumin in saline for 1 h, for example. This would preferably be washed 3 times in in 0.1% bovine serum albumin in saline and then incubated in a secondary antibody directed against the anti-H2S generating enzyme antibody conjugated to horseradish peroxidase, for example, for 30 minutes. After preferably washing 3× in 0.1% bovine serum albumin in saline this would be reacted with tetra-methyl benzidine/0.001% hydrogen peroxide for up to 10 mins or using enhanced chemiluminescence (ECL) with x-ray film. This would be compared to a group of controls (humans who do not have MS) or a pooled control human serum sample and the signal (absorbance/optical density) compared statistically. This is a very inexpensive approach which could be produced in several formats adaptable for clinical or at home use. Variations on the ingredients and steps of the testing kit above which would be obvious to those of ordinary skill in the art are also included in the presently claimed invention. Although a single H2S generating enzyme may be tested with each kit, preferably the kit will include appropriate supplies for testing multiple H2S generating enzymes.

The disclosed embodiments and variations of the present invention have many advantages. Just some of the advantages are described herein. Currently, MM is the most accurate imaging test for the diagnosis and staging of MS. Behavioral and clinical tests also use effective disability scoring (EDSS) which is highly subjective. Additional unbiased and mechanism based testing methods are still needed which can exploit sensitive markers. The use of a combined marker system is much more highly powered and has better statistical utility. The sample that would be analyzed in these tests is a small (<50 μl) blood/serum sample which could be frozen-stored, shipped to a test facility without requiring immediate access to an MRI unit. This approach would lower costs and greatly reduce needs for patient travel and allow for more frequent testing at lower costs. Such samples could be obtained on demand, analyzed using relatively inexpensive supplies and stored frozen until analyzed in clinical labs.

The inventors' disclosed diagnostic method is far less invasive, and requiring only 0.25 μl per protein dot blot, can be easily performed on the side of a routine blood tests, especially for populations who may be suspected to be at risk. The use of the disclosed invention can provide a framework to create an initial snapshot of an individuals' microparticle profile which, inter alia, provides opportunities to compare with later samples and determine how disease is progressing in patients, or to show improvement with therapy to provide an inexpensive and accessible ‘personalized’ medicine approach, or to compare with population averages to allow for early diagnosis or confirmation diagnosis.

The invention includes testing a patient's serum for one, two, or all three of the enzymes (CSE, CBS, and MST). If the serum level of one, two, or all three of a 10-20% or more increase in CSE, a 15-25% or more decrease in CBS, and/or a 15-25% or more decrease in MST, then diagnosing the patient with MS and treating the patient a MS therapeutic. The MS therapeutic may containing one or more of Copaxone, Gilenya, Tecfidera, Tysabri, Aubagio, Rebif, Ampyra, Ocrevus, Avonex, teriflunomide, Plegridy, Mavenclad, Mayzent, ozanimod, Vumerity, Zeposia, prednisone, Betaseron, Avonex Pen, glatiramer, dalfampridine, fingolimod, interferon beta-1a, dexamethasone, dimethyl fumarate, Lemtrada, natalizumab, vedolizumab, steroids, ocrelizumab, Acthar, Extavia, Imuran, valacyclovir Off Label, azathioprine, Dexamethasone Intensol, interferon beta-1b, methylprednisolone, prednisolone, Rebif Rebidose, Solu-Medrol, alemtuzumab, cladribine, Glatopa, mitoxantrone, mycophenolate mofetil Off Label, Azasan, corticotropin, cyclophosphamide Off Label, daclizumab, De-Sone LA, Dxevo, H.P. Acthar Gel, HiDex, Medrol, Millipred, Millipred DP, Orapred ODT, Pediapred, peginterferon beta-1a, siponimod, Bafiertam, diroximel fumarate, Kesimpta, monomethyl fumarate, and ofatumumab.

According to another embodiment, the invention may be a kit comprising materials and reagents for testing for one, two, or three of the enzymes (CSE, CBS, and MST) described herein can be used together with a set of instructions, i.e., to form a kit. The kit may include a therapeutic as described above and instructions for use of the therapeutic.

Products, services or commercial processes or other applications that could result from this invention include Multiple Sclerosis biomarker test kits and therapeutic targets for treatment of MS

A first embodiment of the presently disclosed invention is a noninvasive diagnostic assay (using blood, urine, or saliva, for example) for testing r H2S-generating enzyme presence to diagnosis MS. A second embodiment of the presently disclosed invention is a pharmacological treatment to control MS.

The inventors developed a clinical noninvasive assay testing for H2S-generating enzyme presence to diagnosis MS will increase the accuracy in diagnosis of MS and minimize the cost for the patient.

Additional uses of H2S-generating enzyme in diagnosing or staging disease include the evaluation of H2S-generating enzyme prepared from collected specimens of blood, urine, sputum or saliva.

The terms “inhibition” or “inhibit” refer to a decrease or cessation of any phenotypic characteristic or to the decrease or cessation in the incidence, degree, or likelihood of that characteristic. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents, such as blood serum, blood plasma, Red Blood Cells (RBC), White Blood Cells (WBC), and platelets; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

Pharmaceutical dosage packs comprising one or more containers, each containing one or more doses of an MS therapeutic are also provided. In some embodiments, a unit dosage is provided wherein the unit dosage contains a predetermined amount of a composition comprising an MS therapeutic, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water.

Articles of Manufacture: In some embodiments, an article of manufacture or a kit containing materials useful for the detection of a biomarker (e.g., H2S-generating enzyme) or for the treatment, prevention and/or diagnosis of the disorders described above is provided. The article of manufacture may comprise a container and a label or package insert on or associated with the container. Suitable containers include, for example, bottles, vials, syringes, etc. The containers may be formed from a variety of materials such as glass or plastic. In some embodiments, the container holds a composition which is by itself or combined with another composition effective for treating, preventing and/or diagnosing the condition and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises an MS therapeutic of the invention; and (b) a second container with a composition contained therein, wherein the composition comprises an additional therapeutic agent. The article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. In some embodiments, the article of manufacture may comprise a container with (a) a test for a first H2S-generating enzyme contained therein. In further embodiments, the article of manufacture may comprise the container with (b) a test for a second H2S-generating enzyme contained therein. In further embodiments, the article of manufacture may comprise the container with (c) a test for a third H2S-generating enzyme contained therein. The article of manufacture may further comprise a package insert indicating that the compositions can be used to treat a particular condition and or instructions for conducting the tests, and or instructions for interpreting the results of the tests, such as when levels of the tested H2S-generating enzyme(s) suggest diagnosing a patient with MS. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the molecules of the present invention can be packaged alone or in combination with other therapeutic compounds as a kit. In one embodiment, the other therapeutic compound is second MS therapeutic, distinct from a first MS therapeutic. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Pharmaceutical Compositions: The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methyol, methoxysalicylate, methyloleate, oleic acid, phosphatidylcholine, polyoxyethylene, polysorbate 80, sodium EDTA, sodium glycolate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

The pharmaceutical compositions of the invention may be dispensed to the subject under treatment with the help of an applicator. The applicator to be used may depend on the specific medical condition being treated, amount and physical status of the pharmaceutical composition, and choice of those skilled in the art. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the skin surface, liquid in the container is transferred to the skin in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

The pharmaceutical compositions of the invention may be provided to the subject or the medical professional in charge of dispensing the composition to the subject, along with instructional material. The instructional material includes a publication, a recording, a diagram, or any other medium of expression, which may be used to communicate the usefulness of the composition and/or compound used in the practice of the invention in a kit. The instructional material of the kit may, for example, be affixed to a container that contains the compound and/or composition used in the practice of the invention or shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Other routes of administration to the affected area which are contemplated include: transdermal, mucosal, rectal, and vaginal, or topical (for example, in a carrier vehicle, a topical control release patch, in a wound dressing, a hydrocolloid, a foam, or a hydrogel, a cream, a gel, a lotion, an ointment, a liquid crystal emulsion (LCE), and/or a micro-emulsion). An appropriate biological carrier or pharmaceutically acceptable excipient may be used. Compounds administered may, in various embodiments, be racemic, isomerically purified, or isomerically pure.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

Wherefore, I/we claim:

1. A method of diagnosing Multiple Sclerosis (MS) in a patient comprising:
obtaining a sample from the patient;
determining a level of one or more H2S generating enzymes in the sample from the patient; and
diagnosing the patient with MS when the level of the one or more H2S generating enzymes is one of
at least 15% higher or lower than a control level for the one or more H2S generating enzymes, and
at least 10% higher or lower than a control level for the one or more H2S generating enzymes.

2. The method of claim 1, wherein the patient is diagnosed with MS if one or more H2S generating enzymes is one of Cystathionine g-lyase (CSE), cystathionine b-synthase (CBS), and 3-mercaptopyruvate sulfurtransferase (MST).

3. The method of claim 2, wherein one of
the one or more H2S generating enzymes is CSE, the patient is diagnosed with MS if the level of CSE in the sample is 10-20% higher than a control level,
the one or more H2S generating enzymes is CBS, the patient is diagnosed with MS if the level of CBS in the sample is 15-25% lower than a control level, and
the one or more H2S generating enzymes is MST, the patient is diagnosed with MS if the level of MST in the sample is a 15-25% lower than a control level.

4. The method of claim 2, wherein the patient is diagnosed with MS when both
(a) the one or more H2S generating enzymes includes two of CSE, CBS, and MST, and
(b) two of the following occur
the level of CSE in the sample is 10-20% higher than a control level,
the level of CBS in the sample is 15-25% lower than a control level, and
the level of MST in the sample is a 15-25% lower than a control level.

5. The method of claim 2, wherein the patient is diagnosed with MS when each of
the one or more H2S generating enzymes includes two of CSE, CBS, and MST,
the level of CSE in the sample is 10-20% higher than a control level,
the level of CBS in the sample is 15-25% lower than a control level, and
the level of MST in the sample is a 15-25% lower than a control level.

6. The method of claim 2, wherein the sample is one of plasma, blood, urine, sputum or saliva.

7. The method of claim 6, further comprising centrifugally isolating extracellular vesicles and microparticles from the sample to determine the level of the one or more H2S generating enzymes.

8. The method of claim 7, further comprising centrifuging the sample at 20,000×g for 1 h and analyzing a formed pelleted material for one or more H2S generating enzymes.

9. A method of diagnosing and treating Multiple Sclerosis (MS) in a patient comprising:
obtaining a sample from the patient;
determining a level of one or more H2S generating enzymes in the sample from the patient;
diagnosing the patient with MS when the level of the one or more H2S generating enzymes in the sample is one of
at least 15% higher or lower than a control level for the one or more H2S generating enzymes, and
at least 10% higher or lower than a control level for the one or more H2S generating enzymes, and
administering an effective amount of one of
an H2S generating enzyme inhibitor to the diagnosed patient if the one or more H2S generating enzymes level in the sample is elevated, and
additional H2S generating enzyme substrate and or H2S generating enzyme genetic augmentation to the diagnosed patient if the one or more H2S generating enzymes level in the sample is decreased.

10. The method of claim 9, wherein the sample is one of plasma, blood, urine, sputum or saliva.

11. A method of treating a patient having Multiple Sclerosis (MS) patient in need of treatment comprising;
administering a pharmaceutically effective dose of a therapeutic,
wherein the therapeutic includes one of an H2S generating enzyme inhibitor, additional H2S generating enzyme substrate, and H2S generating enzyme genetic augmentation.

12. The method of claim 11 wherein the one or more H2S generating enzymes includes CSE, and the therapeutic includes a CSE inhibitor.

13. The method of claim 11 wherein the one or more H2S generating enzymes includes CBS, and the therapeutic includes one of a CBS substrate and CBS genetic augmentation.

14. The method of claim 11 wherein the one or more H2S generating enzymes includes MST, and the therapeutic includes one of a MST substrate and MST genetic augmentation.

15. The method of claim 11 wherein
(a) the one or more H2S generating enzymes includes each of CSE, CBS, and MST, and
(b) the therapeutic includes each
(i) a CSE inhibitor,
(ii) one of a CBS substrate and CBS genetic augmentation, and
(iii) one of a MST substrate and MST genetic augmentation.

16. The method of claim 11 further comprising administration of a further therapeutic.

17. The method of claim 16 wherein the further therapeutic includes one of a biologic and immunomodulatory therapy.

18. The method of claim 16 wherein the further therapeutic includes one of Copaxone, Gilenya, Tecfidera, Tysabri, Aubagio, Rebif, Ampyra, Ocrevus, Avonex, teriflunomide, Plegridy, Mavenclad, Mayzent, ozanimod, Vumerity, Zeposia, prednisone, Betaseron, Avonex Pen, glatiramer, dalfampridine, fingolimod, interferon beta-1a, dexamethasone, dimethyl fumarate, Lemtrada, natalizumab, vedolizumab, steroids, ocrelizumab, Acthar, Extavia, Imuran, valacyclovir Off Label, azathioprine, Dexamethasone Intensol, interferon beta-1b, methylprednisolone, prednisolone, Rebif Rebidose, Solu-Medrol, alemtuzumab, cladribine, Glatopa, mitoxantrone, mycophenolate mofetil, Azasan, corticotropin, cyclophosphamide Off Label, daclizumab, De-Sone LA, Dxevo, H.P. Acthar Gel, HiDex, Medrol, Millipred, Millipred DP, Orapred ODT, Pediapred, peginterferon beta-1a, siponimod, Bafiertam, diroximel fumarate, Kesimpta, monomethyl fumarate, and ofatumumab.
